(19) United States
(12) Patent Application Publication
(10) Pub. No.: US 2007/0197983 A1
(43) Pub. Date: Aug. 23, 2007

(54) GENITAL SHIELD AND DIAPER

(76) Inventor: Nicholas William Giles Finn, Edenvale (ZA)

Correspondence Address: DARBY & DARBY P.C., P.O. BOX 770, Church Street Station, New York, NY, 10008-0770, US

(21) Appl. No.: 11/670,503

(22) Filed: Feb. 2, 2007

Related U.S. Application Data
(63) Continuation-in-part of application No. PCT/IB05/02299, filed on Aug. 3, 2005.

(30) Foreign Application Priority Data
Aug. 3, 2004 (ZA) 2004/6186

(51) Int. Cl. A61F 5/44

(52) U.S. Cl. 604/347; 604/385.09; 2/466

(57) ABSTRACT

The invention provides means to support a diaper away from the genitals, particularly for use where surgery has taken place in or around that area. The means takes the form of a genital shield for use with a diaper but may also be a suitably adapted diaper or a diaper which includes such a genital shield. The genital shield is preferably locatable beneath a diaper and has a fluid penetrable semi-rigid body shaped to fit over the genital region and hold the diaper away.

This application is a continuation-in-part of International Patent Application PCT/IB2005/002299 filed Aug. 3, 2005, which claims priority of South African Application No. 2004/6186 filed Aug. 3, 2004, both of which are incorporated by reference herein.

### FIELD OF THE INVENTION

The invention relates to a genital shield for use with a diaper and also to a diaper.

### BACKGROUND OF THE INVENTION

Infant and paediatric circumcision, hypospadias surgery or other similar forms of surgery result in the introduction of a sensitive meatus and/or surgical site to a diaper which becomes the collection receptacle for bodily wastes. These wastes harbour bacteria and pH concentrations harmful to the skin. The result is a post operative complication rate manifesting in infection or the development of meatal ulcers. A proportion of these meatal ulcers further develop into meatal stenoses. The complication rate varies according to literature but is in the range of 0.5% to 10%. There are also reported instances where the complication has resulted in mortality. Most complications require medical management. The essence of the problem is the introduction and continued contact of the exposed meatus and/or surgical site to the diaper which is designed to collect faeces and urine.

The word “diaper” as used in this specification is to be understood in a broad sense including any piece of absorbent material placed around the bottom and between the legs of an infant or incontinent person to absorb bodily waste.

### OBJECT OF THE INVENTION

It is an object of the current invention to provide a genital shield and a diaper that will at least partially overcome the mentioned problem.

### SUMMARY OF THE INVENTION

In accordance with this invention there is provided a genital shield comprising a suitably rigid, fluid penetrable body locatable over the genital region to support a diaper away from the genitals and dimensioned to avoid contact with the genitals.

The invention further provides for the shield to be locatable between a diaper and the genital region.

Further features of the invention provide for the body to be least one layer of liquid absorbent material provided with a semi-rigid form; and for the body to have a base to one side and an opening, for visual alignment of the shield with the genitals, formed through the body at the other side.

Further features of the invention provided for the base to have an upper portion formed with a curved rim locatable against an abdomen and a lower portion locatable below the genitals with two sides of the base extending between the upper and lower portions curved anatomically to correspond to a groin; and for the base to have a lower portion locatable below the genitals and wherein the body adjacent the lower portion of the base is protruded providing a barrier to faecal matter.

A further feature of the invention provides for the shield to have an exterior that carries an adhesive material.

A further feature of the invention provides for the shield to include an at least semi-rigid frame.

Further features of the invention provide for the shield to be for male genitals with a barrier locatable against the groin having an opening through which at least the penis is locatable; for the barrier to be provided from absorbent material; and to include a layer of liquid resistant material, preferably between a pair of absorbent layers.

In accordance with another aspect of the invention there is provided a diaper configured to receive a genital shield as defined above.

Further features of the invention provide for the diaper to be disposable or reusable; and for the shield to be secured to the diaper.

In accordance with yet another aspect of this invention there is provided a diaper which includes a genital shield as defined above.

Further features of the invention provide for the shield to be provided by stiffening of the diaper material and/or by stiffening members.

### BRIEF DESCRIPTION OF THE DRAWINGS

The invention will now be described, by way of example only, with reference to the accompanying drawings which show in

FIG. 1 a perspective view of a genital shield;

FIG. 2 a side view of the genital shield in FIG. 1;

FIG. 3 a bottom view of the genital shield in FIG. 1;

FIG. 4 a perspective view of a second embodiment of a genital shield;

FIG. 5 a cross sectional side view of the genital shield of FIG. 4;

FIG. 6 a perspective view of another genital shield;

FIG. 7 a cross sectional side view of the genital shield of FIG. 6 fitted to an infant male with a reusable diaper;

FIG. 8 a cross sectional side view of the genital shield similar to that of FIG. 6 provided in a disposable diaper and fitted to an infant male; and

FIGS. 9 & 10 perspective views of further embodiments of a genital shield.

### DETAILED DESCRIPTION OF THE INVENTION

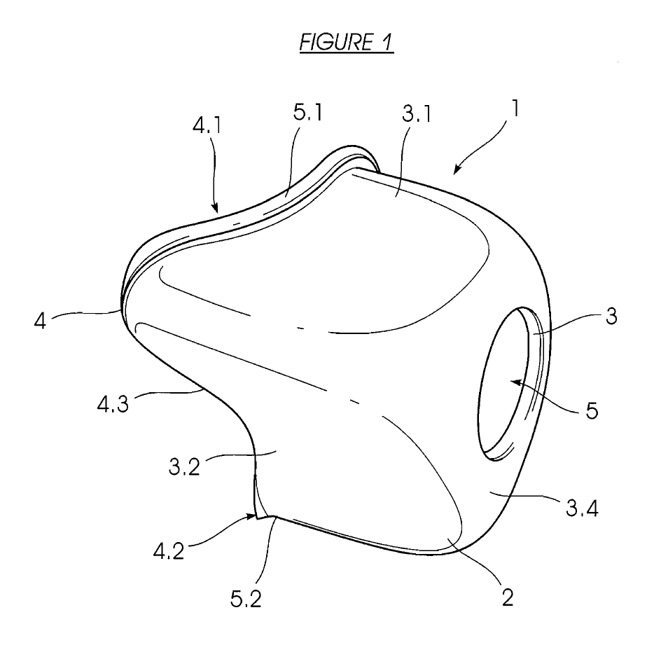

Referring to FIG. 1, a first embodiment of a genital shield (1) in accordance with this invention is shown. The genital shield (1) consists of a body (2) which is shaped to fit over the genital region and support a diaper away from the genitals.

The body (2) is made up of a layer (3) of liquid absorbent material which is provided as a semi-rigid form. The body (2) is hollow with a semi-rigid substantially triangular base (4) to one side. The shield (1) has a domed shape with three walls (3.1, 3.2 and 3.3) extending from the base (4). Supported to the other side of the body (2) is an end wall (3.4). The integral shield (1) is curved to provide a smooth transition between the walls (3.1, 3.2, 3.3 and 3.4). The three sidewalls (3.1, 3.2 and 3.3) are slightly concave.

An opening (5) is provided substantially centrally through the end wall (3.4) of the body (2). The opening (5) allows for visual alignment of the shield (1) with the genitals. This is for proper location of the shield (1) with the base properly spaced around the genitals.

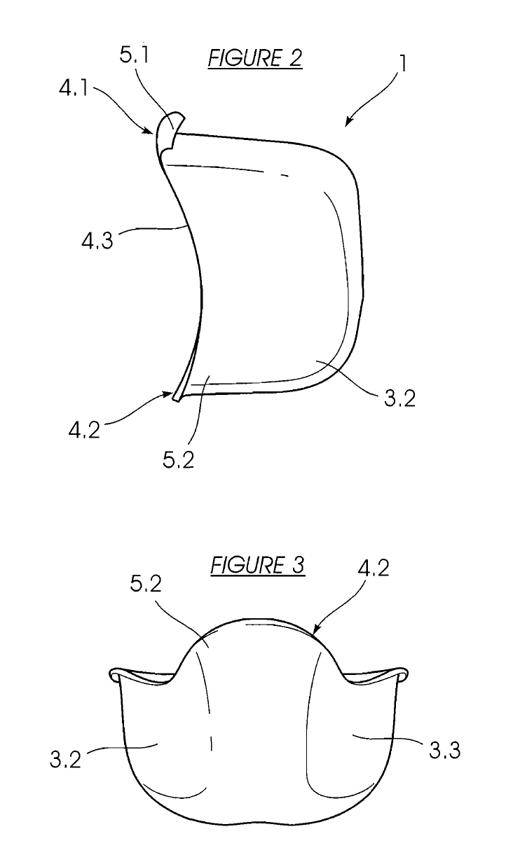

The base (4) has an upper portion (4.1) formed with a curved rim (5.1) that is, in use, locatable above the genitals, against the abdomen. A lower portion (4.2) of the base (4) is locatable below the genitals, between the legs. Two sides (4.3) of the base (4) extend between the upper (4.1) and lower (4.2) portions. These side portions (4.3) are curved anatomically to correspond to the groin. The body (2) adjacent the lower portion (4.2) of the base (2) is protruded at (5.2) to form a rounded extension. The extension (5.2) provides a barrier to faecal matter. This can be seen more clearly in FIGS. 2 and 3.

The semi-rigid form of the shield (1) is provided by a manufacturing process similar to that used for making harmful dust respirators or masks. The layer (3) of material is pressed into the domed shape as seen in the drawings. The material adjacent the edge of the layer (3) may be compressed and treated with a settable adhesive.

The side portions (4.3) of the base (4) may also be provided with a rounded rim, similar to that of the upper portion (4.1).

The size of the sidewalls (3.1, 3.2 and 3.3) is selected to give the shield (1) sufficient height. This is necessary to support the end wall (3.4) away from the genitals. It is common to use a pressure bandage to dress a circumcised penis. This holds the penis erect and elongate. The sidewalls are obviously also suitably spaced apart from each other. The shield (1) is thus dimensioned to avoid contact with the genitals when properly fitted.

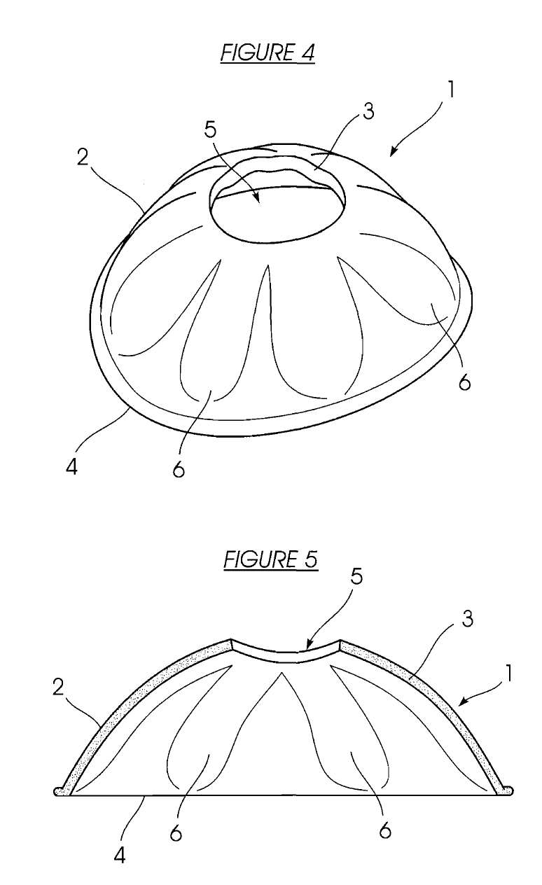

FIGS. 4 and 5 show another embodiment of a genital shield (1). The material and manufacture are similar to that of the first example; however, its design is less refined. A semi-rigid base (4) is provided, together with folds or ridges (6) formed in the layer (3) of material, to give suitable stiffness or rigidity to the body (2). The opening (5) again enables correct positioning of the shield (1) over the genitals, which can be seen therethrough when this is done.

The layer (3) is desirably of a non-woven paper-based material or other suitable material making the shield (1) compliant with the requirements for and qualities of disposable diapers. The shield (1) is fluid penetrable, first absorbing from within and then transmitting to a diaper what will normally be urine.

The exterior of the shield (1) will carry suitable adhesive material (not shown). This is located on the body (2) adjacent to or around the opening (5). The adhesive is covered by a peelable sheet. The sheet is removed once the shield (1) is in place, just before a diaper is fitted. The adhesive sticks to the diaper and deters against movement of the shield (1) from the desired site. When the diaper is checked, the shield (1) simply lifts away from the genitals. The shield (1) remains fixed in its position on the diaper. In the case of a diaper change, the soiled shield (1) need not be handled when the diaper is removed.

The adhesive material is of a gum-like type, suitable for medical applications, and retains effectiveness under damp conditions.

Additional stiffening of the layer (3) of material through suitable treatment or by adding stiffening members (not shown) may also be used. Furthermore, the shield (1) may include a rigid or semi-rigid frame with the absorbent material mounted thereon. The number of layers (3) may vary, and the body (2) may be provided as a composite of layers of different material.

At least part of the underside of the base (4) may be coated with a suitable adhesive, which is preferably covered by a peelable sheet before being used. This adhesive will be of a medically acceptable non-allergenic type, designed for use on skin.

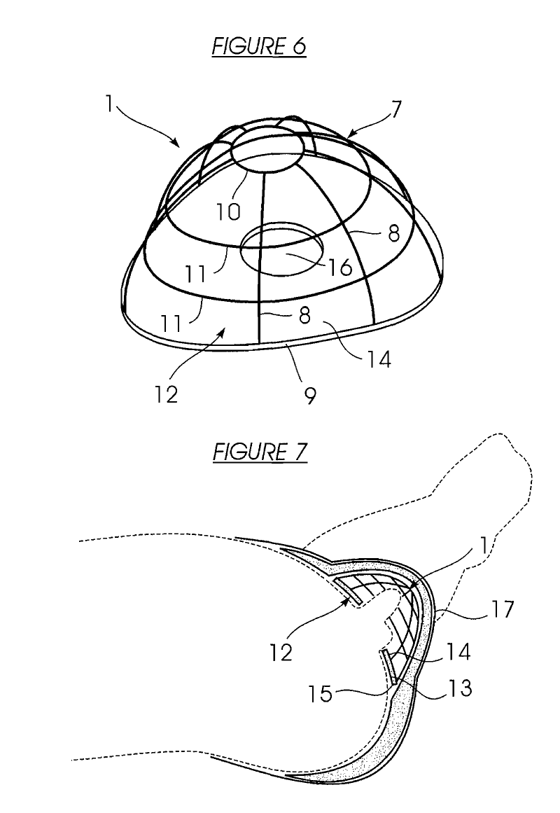

FIGS. 6 to 8 show another embodiment of a genital shield (1). The shield (1) has a body provided by a semi-rigid frame (7) which is generally in the shape of a dome. The frame (7) includes suitably curved ribs (8) extending upwardly from a base (9) and converging at the other side of the frame (7) where their ends are connected to a ring (10). Connecting the ribs (8) at points spaced along their length is a pair of strengthening rings (11).

The base is provided as a rim (9) with a barrier (12) of sheet material secured thereto. The barrier (12) is desirably a composite of absorbent and liquid resistant layers of material. More specifically, the barrier (12) will include a liquid resistant layer (13) between inner (14) and outer (15) layers of absorbent material.

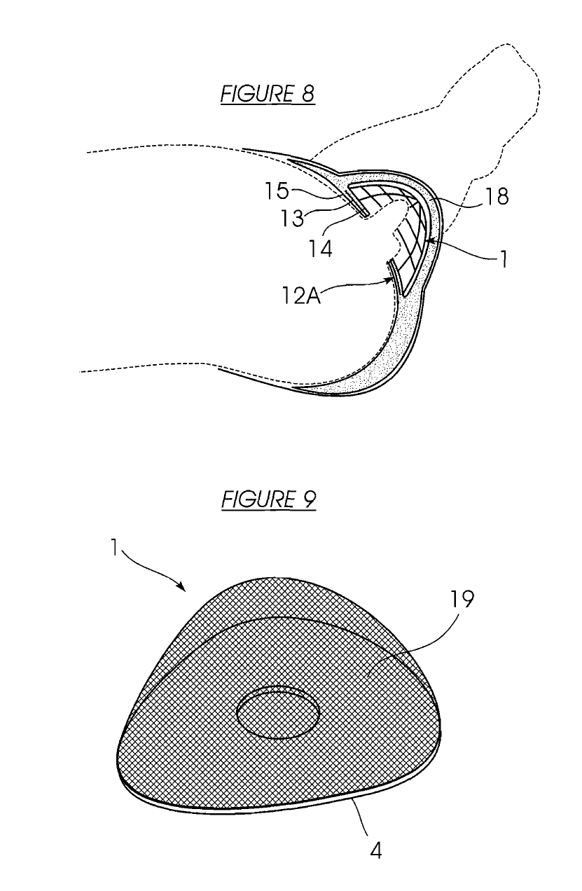

The embodiment of the shield (1) which includes the barrier (12) is for use with male genitals. Accordingly, the barrier (12) has an opening (16) through which at least the penis extends. A larger opening (16) can be provided to accommodate both the penis and scrotum as shown in FIGS. 7 and 8. The opening (16) may take any suitable form and may, for example, be a slit across the barrier (12).

The liquid resistant layer (13) will prevent movement of bodily wastes through the barrier (12). This will prevent urine which is not absorbed by the diaper but by the inner layer (14) of the barrier (12) from passing to the outer layer (15). The liquid resistant layer (7) will thus mitigate contact of urine with the body. The barrier (12) will also restrict faecal contact being made with the male genitalia. While the barrier (12) is shown to be substantially taut it may alternatively be slack or suitably pleated for lateral movement of the opening (16) to accommodate anatomical variances.

The base (9) of the shield (1) will be padded appropriately where this is desired to prevent mechanical chaffing of the skin. The shield (1) can be reusable or disposable. In the case of the former, the barrier (12) will preferably be provided as a disposable removably securable component. One embodiment of such a barrier (12) will be provided with an elasticized border to engage over the rim (9) in use.

The shield (1) can be used separately with a reusable or a disposable diaper (17) as shown in FIG. 7. In this case the shield (1) will be fitted over the genitals and a diaper (17) then secured in place. Alternatively, the shield (1) may be provided as an integral part of a diaper (18) as shown in FIG. 8. In this instance the barrier (12A) has been manufactured as part of a disposable diaper (18) and is secured to extend over the base (9) of the shield (1).

A shield (1) which is provided separately to a diaper (17) can optionally have adhesive on its upper surface where it contacts the diaper (17) and/or adhesive on the base (4) or barrier (12) where it contacts the skin, as already mentioned with regard to previous examples. This will anchor the shield (1) preventing shifting away from optimal positioning. The top adhesive will be similar to that used for woven or non-woven materials and the bottom adhesive may be of the medical kind already mentioned.

This embodiment of the shield (1) will conveniently be moulded from suitable semi-rigid plastics material. It will however be appreciated that other materials and processes of manufacture can also be used. This is to some extent demonstrated in the other examples of the shield (1) that are disclosed.

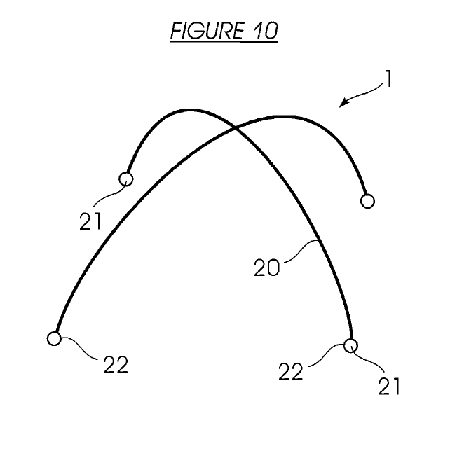

In FIG. 9, a shield (1) is shown with a body (19) provided by gauze. The gauze may be made of wire, plastics material or settable resin. Where such a shield (1) is made from settable materials it may optionally be reinforced by suitable fiber or thread. It will also be appreciated that the shield can be provided by a layer of plastics material moulded to a similar form and having suitable perforations to allow fluid penetration.

FIG. 10 shows a shield (1) provided by four curved ribs (20) secured to each other at one end. The free end of each rib (20) is provided with an enlargement (21) and together these ends define a base (22) for the shield (1).

Unlike the embodiment shown in FIGS. 1 to 5, in the latter embodiments the urine passes directly through the openings in the shield fluid penetrable shield and to the diaper for absorption.

While the diaper (18) in FIG. 8 has the shield (1) as an integral part thereof, a diaper similarly configured to receive a shield (1) may also be provided with the shield (1) removably securable thereto. For example, such a diaper may be formed with a pouch into which the shield (1) is placed. An elasticized hem provided at the outer region of the pouch can be used to retain shields (1) such as those shown in FIGS. 1, 4 and 6 in place. As an alternative, for use with the shield (1) shown in FIG. 10, a diaper (11) can be provided with four suitably spaced pockets with inwardly facing openings. The ribs (8) of the shield (1) will be flexibly resilient and each of the free ends (12) respectively received in the pockets to secure the shield in place.

As a further aspect of the invention, a diaper (not shown) will have an integral genital shield to provide it with a form that fits over the genital region and is supported away from the genitals. Such a shield (1) may be provided by stiffening of the diaper material and/or by stiffening members in or on the diaper. The diaper will thus have a domed formation which fits over the genitals and avoids contact therewith. A suitable stiffening process will vary depending on the material from which the diaper is manufactured. These will include starching and use of settable materials as well as melting into required form. The application of these and other suitable processes to provide such a diaper will be within the competence of a suitably skilled person. The use of stiffening members which may include ribs and rings will also be appreciated.

In use, urine expelled into the shield (1) will largely be absorbed by the diaper which is supported away from the penis. In a specific application, this invention serves to overcome problems following on circumcision or other genital surgery by creating an environment which is preventive of complications for the exposed meatus and surgical site. The dimensions of the shield (1) are selected to prevent contact between the meatus and the shield (1) itself and the diaper which it supports away from the genitals. The usual continued direct contact of the meatus and surgical site with the soiled diaper is avoided. The opportunity for infection and/or the development of meatal ulceration is thus reduced. Also important in this regard is barrier (5.2) which deters against ingress of faecal matter. Additionally the invention creates an environment that is more likely to promote healing.

The shield (1) can be supplied in varying sizes allowing for application to various age groups. It is however anticipated that two sizes will be suitable to cover neo-natal infants and those up to about 1 year of age. The opening (16) in the barrier (12), where it is present, will also be provided in different sizes as required.

Embodiments of the shield (1) from which the barrier (12), shown in FIG. 6, is absent will also find application in protecting female genitalia. It will be appreciated that the dimensions of such a shield (1) may be varied for use with females. It will also be appreciated that in both males and females, the shield (1) may be used in cases less serious than surgical operations or infections, such as rashes and other skin conditions on or around the genitals.

The genital shield may be sterilized and also treated with substances suitable to deter against bacteria. It will further be appreciated that the shape of the shield (1) may be varied but it is anticipated that a generally triangular curved shape for the base (4) will be better suited for location as required over the genitals and part-way between the legs.

While the embodiments of the shield (1) described are generally located between a diaper (11) and the groin area, a shield may also be provided for location on the outer surface of at least one absorbent layer of a diaper, provided the layer or layers on the inside of the shield are supported away from the genitals.

A person suitably skilled in the art will appreciate that a number of other variations which are not specifically mentioned in this description can be made to the genital shield and diaper without departing from the scope of this invention.

1. A genital shield comprising a suitably rigid, fluid penetrable body locatable over the genital region to support a diaper away from the genitals and dimensioned to avoid contact with the genitals.

2. A shield as claimed in claim 1 which is locatable between a diaper and the genital region.

3. A shield as claimed in claim 2 in which the body is at least one layer of liquid absorbent material provided with a semi-rigid form.

4. A shield as claimed in claim 3 having a base to one side and an opening, for visual alignment of the shield with the genitals, formed through the body at the other side.

5. A shield as claimed in claim 4 in which the base has an upper portion formed with a curved rim locatable against an abdomen.

6. A shield as claimed in claim 4 in which the base has an upper portion locatable against an abdomen and a lower portion locatable below the genitals with two sides of the base extending between the upper and lower portions curved anatomically to correspond to a groin.

7. A shield as claimed in claim 4 in which the base has a lower portion locatable below the genitals and wherein the body adjacent the lower portion of the base is protruded providing a barrier to faecal matter.

8. A shield as claimed in claim 3 in which an exterior of the shield carries an adhesive material.

9. A diaper configured to receive a genital shield as claimed in claim 1.

10. A diaper as claimed in claim 9 in which the shield is secured to the diaper.

11. A diaper that includes a genital shield as claimed in claim 1.

12. A diaper as claimed in claim 11 in which the shield is provided by stiffening of the diaper material.

13. A diaper as claimed in claim 11 in which the shield is provided by stiffening members.
